(19) United States
(12) Patent Application Publication
(10) Pub. No.: US 2023/0065784 A1
(43) Pub. Date: Mar. 2, 2023

(54) COMPOSITION AND METHOD FOR REDUCING EXPRESSION OF CHECKPOINT INHIBITORS IN T CELLS EXPRESSING A CAR OR CTL

(71) Applicant: City of Hope, Duarte, CA (US)

(72) Inventors: Saul J. Priceman, Duarte, CA (US); John C. Burnett, Duarte, CA (US); Yukiko Yamaguchi, Duarte, CA (US); Elizabeth Epps, Duarte, CA (US)

(21) Appl. No.: 17/440,832

(22) PCT Filed: Mar. 20, 2020
(86) PCT No.: PCT/US2020/023970

§ 371 Date: Sep. 20, 2021

Related U.S. Application Data
(60) Provisional application No. 62/821,923, filed on Mar. 21, 2019.

(51) Int. Cl. C07K 14/725; C07K 16/28; C12N 15/63; C12N 15/113

(52) U.S. Cl. CPC C07K 14/7051; C07K 16/2818; C12N 15/63; C12N 15/113

(57) ABSTRACT

Described herein is an approach for targeting multiple critical checkpoint genes involved in T cell exhaustion, for example, PD-1, TIM3, and LAG-3 in a manner that allows knock-down of their expression in T cells that also express a T cell receptor targeted to cancer cells, e.g., a CAR targeted to a cancer antigen or a T cell receptor (TCR).

### CROSS-REFERENCE TO RELATED APPLICATIONS

This application is a U.S. National Phase Application under 35 U.S.C. § 371 of International Application No. PCT/US2020/023970, filed on Mar. 20, 2020, which claims priority to and the benefit of U.S. Provisional Application No. 62/821,923, filed on Mar. 21, 2019. The entire contents of the foregoing are incorporated herein by reference.

### SEQUENCE LISTING

The instant application contains a Sequence Listing which has been submitted electronically in ASCII format and is hereby incorporated by reference in its entirety. Said ASCII copy, created on Sep.15, 2021, is named SequenceListing.txt and is 48,000 bytes in size.

### BACKGROUND

Tumor-specific T cell based immunotherapies, including therapies employing engineered T cells, have been investigated for anti-tumor treatment.

Adoptive T cell therapy, including chimeric antigen receptor (CAR) T cell therapy, has come to the forefront of immunotherapy approaches for multiple diseases, including cancer and HIV. Clinical and preclinical data indicate that exhausted T cells, through activation of immune checkpoint pathways, may render adoptive therapy incapable of their full potential. Therefore, several strategies to improve persistence and functionality of T cells have been investigated. Immune checkpoint pathways, including the programmed cell death protein-1 (PD-1) and the cytotoxic T lymphocyte-associated protein-4 (CTLA4), have emerged as critical drivers of immunosuppression in solid cancers, by limiting both adaptive anti-tumor immunity as well as adoptive T cell therapies. Nivolumab, an anti-PD-1 antibody, and ipilimumab, an anti-CTLA4 antibody, are both FDA-approved for advanced melanoma, and are under intense investigation for other metastatic cancers alone and in combination. However, several issues have highlighted the need for additional therapeutic intervention to improve functionality of T cells.

### SUMMARY

Described herein is an approach for targeting multiple critical checkpoint genes involved in T cell exhaustion, for example, PD-1, TIM3, and LAG-3 in a manner that allows knock-down of their expression in T cells that also express a T cell receptor targeted to cancer cells, e.g., a CAR targeted to a cancer antigen or a T cell receptor (TCR). These genes have been independently implicated in T cell exhaustion, and several clinical antibodies have been engineered to block protein function. In the approach detailed herein, siRNA is used to knock-down multiple checkpoint genes simultaneously in T cells engineered for improved therapeutic responses. As described below, shRNA targeted to PD-1, TIM-3, and LAG-3 were incorporated them into a lentiviral cassette under the control of independent polymerase 3 promoters.

Described herein is a nucleic acid vector comprising an shRNA sequence targeted to an mRNA encoding a checkpoint inhibitor selected from the group consisting of PD-1, TIM3 and LAG3 and a nucleic acid sequence encoding a chimeric antigen receptor (CAR).

In various embodiments: the CAR targets an antigen selected from the group consisting of IL-13Ra, HER2, CD19 and PSCA; each shRNA sequence is operably linked to a promoter; each shRNA sequence is operably linked to the same promoter; the vector comprises two or more different shRNA nucleic acid sequences targeted to the same mRNA encoding a checkpoint inhibitor; each shRNA sequence is targeted to a different mRNA encoding a checkpoint inhibitor; the vector comprises an shRNA sequence targeted to an mRNA encoding PD-1 and an shRNA sequence targeted to an mRNA encoding TIM3; the vector comprises an shRNA sequence targeted to an mRNA encoding PD-1 and an shRNA sequence targeted to an mRNA encoding LAG3; the vector comprises an shRNA sequence targeted to an mRNA encoding TIM3 and an shRNA sequence targeted to an mRNA encoding LAG3; each shRNA sequence is operably linked to a different promoter; the vector comprises two or more different shRNA sequences that are operably linked to the same promoter; the promoter is selected from the group consisting of an H1 DNA POL III promoter, an U6 DNA POL III promoter, and a 75K DNA POL III; the shRNA targeted to an mRNA encoding PD-1 is targeted to SEQ ID NO:25; the shRNA targeted to an mRNA encoding TIM3 is targeted to SEQ ID NO:26; the shRNA targeted to an mRNA encoding LAG3 is targeted to SEQ ID NO:27; the shRNA targeted to PD-1 comprises a nucleic acid sequence selected from the group consisting of SEQ ID Nos 12-14; the shRNA targeted to TIM3 comprises a nucleic acid sequence selected from the group consisting of SEQ ID Nos: 15-17; the shRNA targeted to LAG3 comprises a nucleic acid sequence selected from the group consisting of SEQ ID Nos: 18-20; each shRNA sequence comprises: sense sequence and an antisense sequence, wherein the sense and the antisense sequence, wherein the sense sequence comprises a nucleotide sequence identical to a target sequence in an mRNA encoding a checkpoint inhibitor; and the vector is a lentiviral vector.

Described herein is a nucleic acid vector comprising an H1 DNA POL III promoter, an U6 DNA POL III promoter, and a 75K DNA POL III promoter, where each promoter is operably linked to an shRNA nucleic acid sequence targeted to an mRNA encoding a checkpoint inhibitor.

In various embodiments: each shRNA nucleic acid sequence is targeted to the same mRNA encoding a checkpoint inhibitor; each shRNA is targeted to a different mRNA encoding a checkpoint inhibitor; the checkpoint inhibitor is selected from the group consisting of PD-1, TIM3 and LAG3; the shRNA targeted to an mRNA encoding PD-1 is targeted to SEQ ID NO:25; the shRNA targeted to an mRNA encoding TIM3 is targeted to SEQ ID NO:26; the shRNA targeted to an mRNA encoding LAG3 is targeted to SEQ ID NO:27; the shRNA targeted to PD-1 comprises a nucleic acid sequence selected from the group consisting of SEQ ID Nos 12-14; the shRNA targeted to TIM3 comprises a nucleic acid sequence selected from the group consisting of SEQ ID Nos: 15-17; the shRNA targeted to LAG3 comprises a nucleic acid sequence selected from the group consisting of SEQ ID Nos: 18-20; the vector comprises the nucleotide sequence of SEQ ID NO:24; each shRNA sequence comprises: sense sequence and an antisense sequence, wherein the sense and the antisense sequence, wherein the sense sequence comprises a nucleotide sequence identical to a target sequence in an mRNA encoding a checkpoint inhibitor; the vector is a lentiviral vector; the vector further comprises a nucleotide sequence encoding a chimeric antigen receptor (e.g., PSCA, HER2 or CD19).

Also disclosed is a T cell harboring the nucleic acid vector as described herein. Also disclosed is a method for reducing the expression of a checkpoint inhibitor in T cell, the method comprising introducing a nucleic acid vector as described herein into the T cell.

### DESCRIPTION OF DRAWINGS

FIG. 1 depicts the results of an experiment showing that anti-PD1 antibody, Nivolumab, induces compensatory upregulation of TIM3 in T cells. CD4+ T cells were co-cultured with in vitro differentiated CD14+ dendritic cells, and treated with 0.1, 1, 10 μg/mL of Nivolumab. After 5 days, T cells were analyzed by flow cytometry for cell surface expression of exhaustion markers, PD-1, TIM3, LAG3, and CTLA-4.

FIG. 2 depicts the results of a study assessing the impact of PSCA-targeted CAR on advanced prostate cancer. (a) Illustration of PSCA-CAR design with PSCA(ΔCH2)BBζ with an anti-PSCA scFv tethered to the membrane by a modified IgG4 Fc linker (deleted CH2 domain), containing a CD4 transmembrane domain, an intracellular 4-1BB co-stimulatory domain and a CD3ζ cytolytic domain. The T2A skip sequence separates the CAR from a truncated CD19 (CD19t) protein for cell tracking. (b-c) NSG male mice bearing PC-3 (c) and DU145 (d) tumors over-expressing human PSCA were treated with a single intravenous dose of 1, 2.5 or 5×106 PSCA(ΔCH2)BBζ CAR or 5×10⁶ Mock (untransduced) T cells, and tumor volume was monitored by caliper measurement. (d-e) PC-3-PSCA (d) and DU145-PSCA (e) tumors were stained for CD3 (human T cells), PD-1, and PD-L1 expression by immunohistochemistry, and representative images (20× magnification) are shown from Mock (untransduced) and PSCA (ΔCH2)BBζ CAR T cell-treated mice.

FIG. 3 depicts the amino acid sequence of the PSCA CAR used in the studies described herein.

FIG. 4 depicts the results of a study assessing the impact of anti-PD-1 antibody blockade in combination with PSCA CAR T cells in vitro. (a) IFNγ production quantified by ELISA in supernatants from Mock (untransduced) or PSCA-CAR T cells cultured overnight with DU145 or DU145-PSCA tumor cells in the presence or absence of indicated concentrations of anti-human PD-1 antibody Nivolumab. (b) DU145-PSCA tumor killing (left), and PD-1 (middle) and 4-1BB expression in PSCA-CAR T cells (right), following a 4 and 8 day co-culture in the presence or absence of Nivolumab. (c) NSG male mice bearing DU145-PSCA tumors were treated with a single intravenous dose of 1×10⁶ PSCA(ΔCH2)BBζ CAR or Mock (untransduced) T cells, and treated with 4 doses with 100 ug Nivolumab, and tumor volume was monitored by caliper measurement. (d) Percentage of human CD3+ cells (top) and PD-1 expression in tumor-infiltrating T cells from mice treated with PSCA-CAR T cells alone or with Nivolumab.

FIG. 5 depicts the results of a study examining the impact of M1 and M2 macrophages on PD-L1 expression and PSCA-CAR T cell mediated anti-tumor responses in vitro (a) Schematic of assay used. Macrophages, CAR T cells, and tumors were co-cultured for 6 or 10 days, and evaluated for functional assays using flow cytometry. (b) Representative bright field images of M1 and M2 macrophages. (c) Phenotypes of M1 and M2 macrophages using indicated cell surface markers, evaluated by flow cytometry. (d) CAR T cell killing of DU145-PSCA tumor cells in the presence or absence of M1 or M2 macrophages. (e) Mock (untransduced) and PSCA-CAR T cell proliferation and activation (indicated by expression of 4-1BB) in the presence or absence of M1 and M2 macrophages. (f) PD-L1 expression in DU145-PSCA tumor cells and M1 or M2 macrophages following co-culture with Mock or PSCA-CAR T cells.

FIG. 6 depicts the results of a study examining the impact of the HUSKY vector. (a) Diagram of pHIV7-backbone with HUSKY cassette and GFP reporter. (b) HEK293T cells were co-transfected with a dual luciferase plasmid (Firefly and Renilla) and one of three HUSKY cassette vectors containing shRenilla driven by H1, U6, or 7SK promoters. Empty HUSKY was used as a negative control. Cells were analyzed for luminescence signal 48 hours post-transfection and the Renilla/Firefly ratio determined. Results with p<0.05 were considered significant by one-way ANOVA analysis N=3.

FIG. 7 depicts the results of an analysis of HUSKY-mediated knockdown of PD1, TIM3, and LAG3. HEK293T cells were co-transfected with a dual luciferase plasmid containing either the 100 bp sense or antisense targets for each shRNA in the Renilla 3′UTR and a HUSKY vector containing one of three candidate 7SK-driven shPD1, H1-driven shTIM3, or U6-driven shLAG3. HUSKY-shRenilla vectors for each promoter were used as positive controls and empty HUSKY as a negative control. Sense vs. antisense target strand knockdown was assessed. Cells were analyzed for luminescence signal 48 hours post-transfection and the Renilla/Firefly ratio determined. Results with p<0.05 were considered significant by one-way ANOVA analysis. N=6.

FIG. 8 depicts the results of an examination of knock-down of PD-1 by HUSKY shRNA constructs in HEK 293T-PD-1 cells. PD-1 and GFP were overexpressed in 293T cells by lentivirus transduction, and double positive cells were obtained by FACS. HEK 293T-PD-1 cells were used to screen shRNA candidates against PD-1 by Lipofectamine transfection of plasmids with shRNA candidates on day 0, and on day 5, PD-1 expression was evaluated by flow cytometry.

FIG. 9 depicts the nucleotide sequence of the HUSKY cassette without any snRNA inserts SEQ ID NO: 24). The H1 (SEQ ID NO: 21), U6 (SEQ ID NO: 22) and 7SK (SEQ ID NO:23) promoters are indicated with italics and underlining.

### DETAILED DESCRIPTION

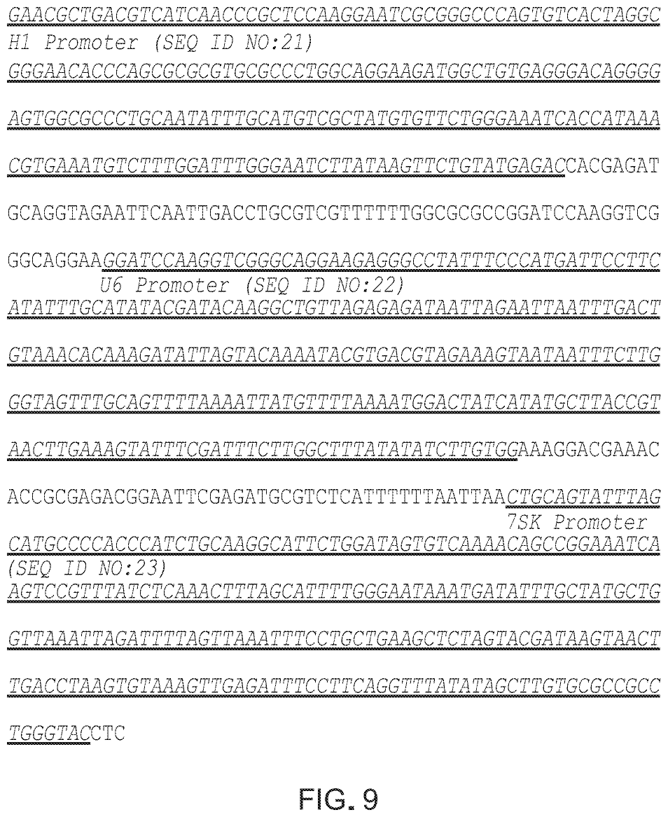

An example of a HUSKY cassette with three promoters for expression of shRNA targeted to checkpoint inhibitors is depicted in FIG. 9.

A HUSKY cassette can include and shRNA sequence targeted to Human PD-1 (Genbank NM_005018) mRNA (SEQ ID NO:25).

The HUSKY cassette can include and shRNA sequence targeted to Human TIM3 (Genbank NM_032782) mRNA (SEQ ID NO: 26).

The HUSKY cassette can include and shRNA sequence targeted to Human LAG3 (Genbank NM_002286) mRNA (SEQ ID NO:27).

HUSKY cassette can be used to reduce expression of one or more checkpoint inhibitors, e.g., one or more (e.g., all) of PD-1, TIM3 and LAG3 in T cells, e.g., T cells expressing a CAR. Reduced expression of one or of PD-1, TIM3 and LAG3 can be useful in conjunction with expression of a CAR or a TCR targeted to a cancer antigen, for example, PSCA, CD19 or HER2. Suitable CAR include, but are not limited to, those described in: WO 2017/079694 (HER2); WO 2017/062628 (PSCA); and US 2016/0340649 (IL-13Ralpha2). Each CAR includes a targeting sequence, which can e an svFv or a receptor ligand; a spacer sequence, a transmembrane domain, a co-stimulatory domain and a CD3 zeta domain. Examples of each are provided below.

### scFv Sequences

A variety of scFv targeting sequences can be used CAR. Suitable sequences for targeting PSCA

include:
(SEQ ID NO: 28)
DIQLTQSPSTLSASVGDRVTITCSASSSVRFIHWYQQKPGKAP
KRLIYDTSKLASGVPSRFSGSGSGTDFTLTISSLQPEDFATYY
CQQWGSSPFTFGQGTKVEIKGSTSGGGSGGGSGGGGSSEVQLV
EYGGGLVQPGGSLRLSCAASGFNIKDYYIHWVRQAPGKGLEWV
AWIDPENGDTEFVPKFQGRATMSADTSKNTAYLQMNSLRAEDT
AVYYCKTGGFWGQGTLVTVSS.

Suitable sequences for targeting CD19 include:

FMC63 scFv:
(SEQ ID NO: 29)
IPDIQMTQTTSSLSASLGDRVTISCRASQDISKYLNWYQQ
KPDGTVKLLI YHTSRLHSGV PSRFSGSGSGTDYSLTIS
NLEQEDIATYFCQQGNTLPYTFGGGTKLEITGSTSGSGKP
GSGEGSTKGEVKLQESGPGLVAPSQSLSVTCTVSGVSLPD
YG VSWIRQPPRKGLEWLGVIWGSETTYYNSALKSRLTII
KDN SKSQVFLKMN SLQTDDTAIYYCAKHYYYGGSYAMD
YWGQGTSVTVSS.
FMC63 VL:
(SEQ ID NO: 30)
DIQMTQTTSSLSASLGDRVTISCRASQDISKYLNWYQQKP
DGTVKLLIYHTSRLHSGVPSRFSGSGSGTDYSLTISNLEQ
EDIATYFCQQGNTLPYTFGGGTKLEIT.
FMC63 VH:
(SEQ ID NO: 31)
EVK LQESGPGLVAPSQSLSVTCTVSGVSLPDYGVSWIRQ
PPRKGLEWLGVIWGSETTYYNSALKSRLTIIKDN SKSQV
FLKMNSLQTDDTAIYYCAKHYYYGGSYAMDYWGQGTSVTV
SS.

Additional scFv that bind CD19 are described in US 2016/0152723 and in WO 2016/033570.

### Spacer Region

The CAR can include a spacer located between the targeting domain (e.g., the scFv) and the transmembrane domain. A variety of different spacers can be used. Some of them include at least portion of a human Fc region, for example a hinge portion of a human Fc region or a CH3 domain or variants thereof. Table 1 below provides various spacers that can be used.

TABLE 1
Examples of Spacers

| Name | Length | Sequence |
|---|---|---|
| a3 | 3 aa | AAA |
| linker | 10 aa | GGGSSGGGSG (SEO ID NO: 32) |
| IgG4 hinge (S→P) | 12 aa | ESKYGPPCPPCP (SEQ ID NO: 33) |
| (S228P) | | |
| IgG4 hinge | 12 aa | ESKYGPPCPSCP (SEQ ID NO: 34) |
| IgG4 hinge | 22 aa | ESKYGPPCPPCPGGGSSGGGSG (SEQ ID NO: 35) |
| (S228P) + linker | | |
| CD28 hinge | 39 aa | IEVMYPPPYLDNEKSNGTIIHVKGKHLCPSPLFPGPSKP |
| | | (SEQ ID NO: 36) |
| CD8 hinge-48aa | 48 aa | AKPTTTPAPRPPTPAPTIASQPLSLRPEACRPAAGGAVH |
| | | TRGLDFACD (SEQ ID NO: 37) |
| CD8 hinge-45aa | 45 aa | TTTPAPRPPTPAPTIASQPLSLRPEACRPAAGGAVHTRGLDFACD |
| | | (SEQ ID NO: 38) |
| IgG4(HL-CH3) | 129 aa | ESKYGPPCPPCPGGGSSGGGSGGQPREPQVYTLPPSQEEMTKNQV |
| (includes S228P | | SLTCLVKGFYPSDIAVEWESNGQPENNYKTTPPVLDSDGSFFLYS |
| in hinge) | | RLTVDKSRWQEGNVFSCSVMHEALHNHYTQKSLSLSLGK |
| | | (SEQ ID NO: 39) |
| IgG4 | 229 aa | ESKYGPPCPSCPAPEFEGGPSVFLFPPKPKDTLMISRTPEVTCVVV |
| (L235E, N2970) | | DVSQEDPEVQFNWYVDGVEVHQAKTKPREEQFQSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 40) |
| IgG4(S228P, | 229 aa | ESKYGPPCPPCPAPEFEGGPSVFLFPPKPKDTLMISRTPEVTCVVV |
| L235E, N2970) | | DVSQEDPEVQFNWYVDGVEVHQAKTKPREEQFQSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 41) |
| IgG4(CH3) | 107 aa | GQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHE |
| | | ALHNHYTQKSLSLSLGK (SEQ ID NO: 42) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:34) or ESKYGPPCPPCP (SEQ ID NO:33).

The hinge/linger region can also comprise the sequence ESKYGPPCPPCP (SEQ ID NO:33) followed by the linker sequence GGGSSGGGSG (SEQ ID NO:32) followed by IgG4 CH3 sequence GQPREPQVYTLPP SQEEMTKNQVSLTCLVKGFYP SDIAVEWESNGQPENNYKTTPPVLDSDGSFFLYSRLTVDKSRWQEGNVFSCSVMHEALHNHYTQKSLSLSLGK (SEQ ID NO:41). Thus, the entire linker/spacer region can comprise the sequence: ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK (SEQ ID NO:______) In some cases, the spacer has 1,2,3,4, or 5 single amino acid changes (e.g., conservative changes) compared to SEQ ID NO:______. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in CAR. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 43) |
| CD28 | NM_006139 | 27 aa | FWVLVVVGGVLACYSLLVTVAFII |
| | | | FWV (SEQ ID NO: 44) |
| CD28(M) | NM_006139 | 28 aa | MFWVLVVVGGVLACYSLLVTVAFI |
| | | | IFWV (SEQ ID NO: 45) |
| CD4 | M35160 | 22 aa | MALIVLGGVAGLLLFIGLGIFF |
| | | | (SEQ ID NO: 46) |
| CD8tm | NM_001768 | 21 aa | IYIWAPLAGTCGVLLLSLVIT |
| | | | (SEQ ID NO: 47) |
| CD8tm2 | NM_001768 | 23 aa | IYIWAPLAGTCGVLLLSLVITLY |
| | | | (SEQ ID NO: 48) |
| CD8tm3 | NM_001768 | 24 aa | IYIWAPLAGTCGVLLLSLVITLYC |
| | | | (SEQ ID NO: 49) |
| 41BB | NM_001561 | 27 aa | IISFFLALTSTALLFLLFF |
| | | | LTLRFSVV |
| | | | (SEQ ID NO: 50) |

### Costimulatory and CD3zeta Domain

The costimulatory domain can be any domain that is suitable for use with a CD3ζ signaling domain. In some cases, the costimulatory domain is a CD28 costimulatory domain that includes a sequence that is at least 90%, at least 95%, at least 98% identical to or identical to: RSKRSRGGHSDYMNMTPRRPGPTRKHYQPYAPPRDFAAYRS (SEQ ID NO:52; LL to GG amino acid change double underlined). In some cases, the CD28 co-signaling domain has 1, 2, 3, 4 of 5 amino acid changes (preferably conservative and preferably not in the underlined GG sequence) compared to SEQ ID NO:23. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:54). In some cases, the 4-1BB co-signaling domain has 1, 2, 3, 4 or 5 amino acid changes (preferably conservative) compared to SEQ ID NO:54.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples
of Costimulatory Domains

| Name | Accession | Length | Sequence |
|---|---|---|---|
| CD3ζ | J04132.1 | 113 aa | RVKFSRSADAPAYQQGQNQLYNELNL |
| | | | GRREEYDVLDKRRGRDPEMGGKPRRK |
| | | | NPQEGLYNELQKDKMAEAYSEIGMKG |
| | | | ERRRGKGHDGLYQGLSTATKDTYDAL |
| | | | HMQALPPR (SEQ ID NO: 51) |
| CD28 | NM_006139 | 42 aa | RSKRSRLLHSDYMNMTPRRPGPTRKH |
| | | | YQPYAPPRDFAAYRS |
| | | | (SEQ ID NO: 52) |
| CD28gg* | NM_006139 | 42 aa | RSKRSRGGHSDYMNMTPRRPGPTRKH |
| | | | YQPYAPPRDFAAYRS |
| | | | (SEQ ID NO: 53) |
| 4-1BB | NM_001561 | 42 aa | KRGRKKLLYIFKQPFMRPVQTTQEED |
| | | | GCSCRFPEEEEGGCEL |
| | | | (SEQ ID NO: 54) |
| OX40 | | 42 aa | ALYLLRRDQRLPPDAHKPPGGGSFRT |
| | | | PIQEEQADAHSTLAKI |
| | | | (SEQ ID NO: 55) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and in some cases a short linker consisting of 2-10, e.g., 3 amino acids (e.g., GGG) is positioned between the costimulatory domain and the CD3ζ signaling domain.

### CD3ζ Signaling Domain

The CD3ζ Signaling domain can be any domain that is suitable for use with a CD3 ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEGLYNELQKDKMAEAYSEIGMKGERRRGKGRDGLYQGLSTATKDTYDALHMQALPPR (SEQ ID NO:51). In some cases, the CD3ζ signaling has 1, 2, 3, 4 of 5 amino acid changes (preferably conservative) compared to SEQ ID NO:51.

### Truncated EGFR

The CD3ζ signaling domain can be followed by a ribosomal skip sequence (e.g., LEGGGEGRGSLLTCGDVEENPGPR; SEQ ID NO:56) and a truncated EGFR having a sequence that is at least 90%, at least 95%, at least 98% identical to or identical to: LVTSLLLCELPHPAFLLIPRKVCNGIGIGEFKDSLSINATNKHFKNCTSISGDLHILPVAFRGD SFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRG PDNCIQCAHYIDGPHCVKTCPAGVMGENNTLVWKYADAGHVCHLCHPNCTYGCTGPGLEGCPTNGPKIPSIATGMVGALLLLLVVALGIGLFM (SEQ ID NO:57). In some cases, the truncated EGFR has 1, 2, 3, 4 of 5 amino acid changes (preferably conservative) compared to SEQ ID NO:57.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

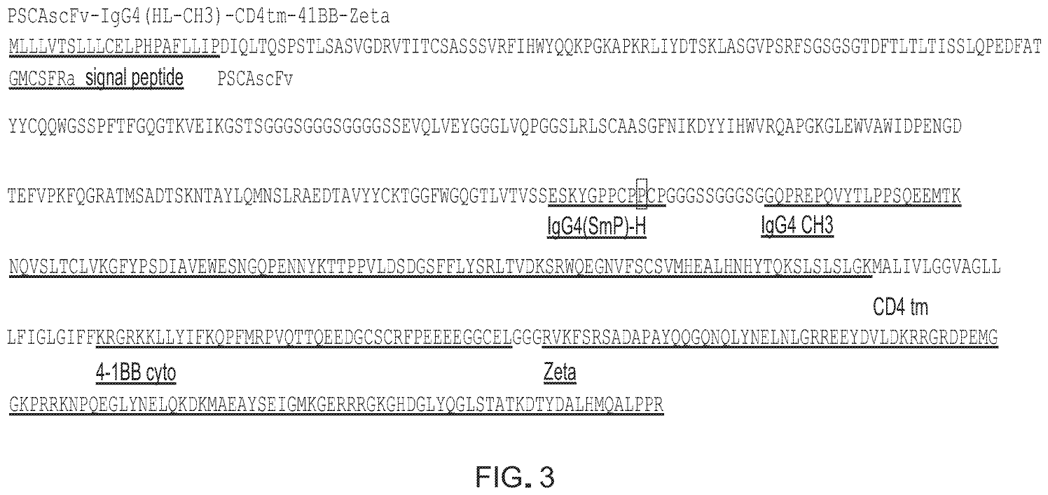

The CAR can include a sequence that is at least 90%, at least 95%, at least 98% identical to or identical to the amino acid sequence depicted in FIG. 3 (SEQ ID Nos:______), either including or excluding the GMCSFRa signal sequence and either including or excluding the T2A ribosomal skip sequence and the truncated EGFRt).

In some cases, the CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the CAR lentiviral vector can act as suicide gene to ablate the CAR+ T cells in cases of treatment-related toxicity.

The vectors can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of the CAR as well as a non-immunogenic surface marker for in vivo detection, ablation, and potential ex vivo selection. The activated/genetically modified central memory T cells can be expanded in vitro with IL-2/IL-15 and then cryopreserved.

### EXAMPLES

### Example 1: Impact of Targeting PD1 on Expression of Checkpoint Inhibitors

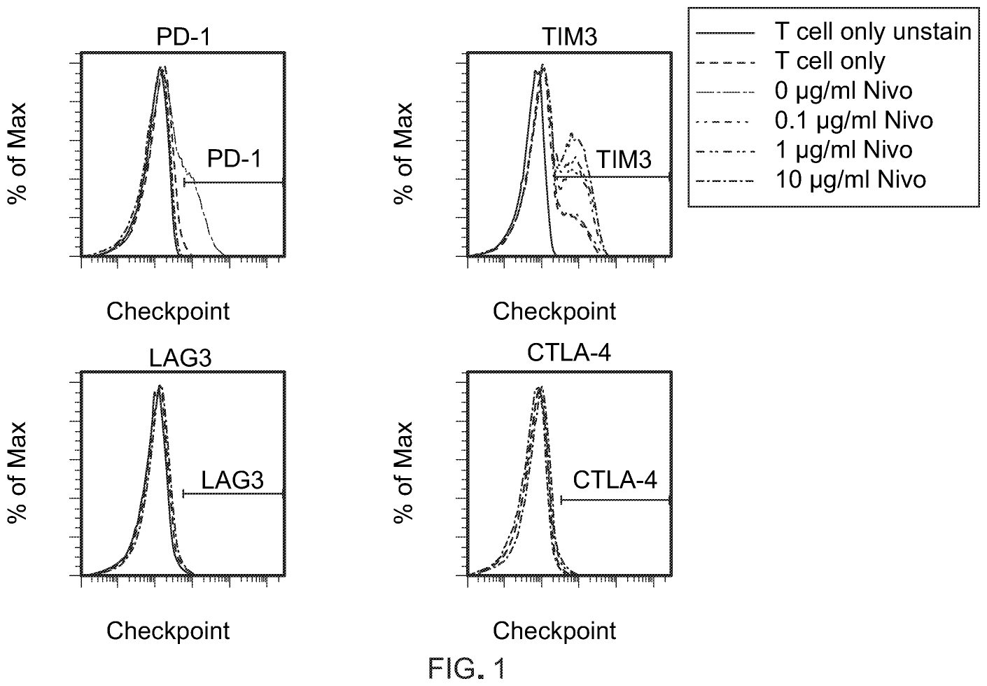

Efforts to target a checkpoint inhibitor can lead to upregulation of one or more other checkpoint inhibitors. Nivolumab, an anti-PD1 antibody can cause upregulation of TIM3. In this study, the results of which are depicted in FIG. 1, CD4+ T cells were co-cultured with in vitro differentiated CD14+ dendritic cells, and treated with 0.1, 1, 10 μg/mL of Nivolumab. After 5 days, T cells were analyzed by flow cytometry for cell surface expression of exhaustion markers, PD-1, TIM3, LAG3, and CTLA-4.

### Example 2: Evaluation of Anti-PD-1 Antibody Blockade in Combination with PSCA CAR T Cells In Vitro and In Vivo

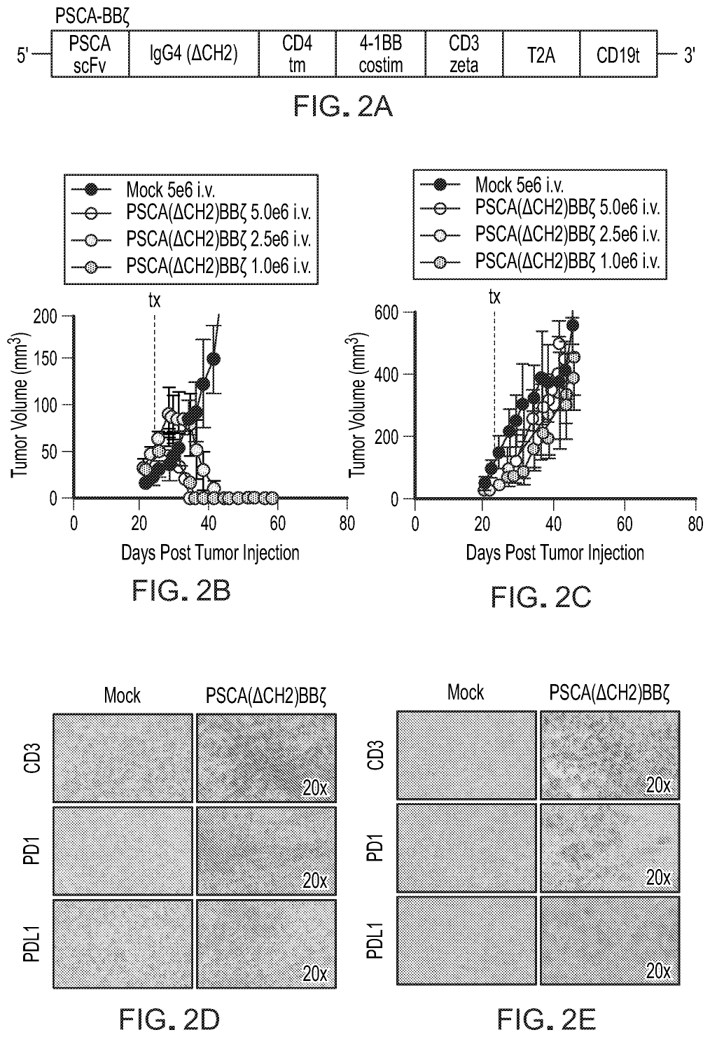

A CAR (PSCA(ΔCH2)BBζ) CAR targeting PSCA was developed. This CAR is depicted schematically in FIG. 2A. The PSCA(ΔCH2)BBζ includes: the MB1 scFv targeting PSCA, IgG4(HL-CH3) spacer, lacks the CH2 domain and has a short linker sequence located between the hinge region and the CH3 region, a CD4 transmembrane domain, the 4-1BB co-stimulatory domain and the CD3ζ cytoplasmic signaling domain. The CAR coding sequence is followed by the T2A ribosomal skip sequence and a truncated CD19 sequence to permit co-expression of surface, signaling incompetent, truncated CD19 as a marker. The complete amino acid sequence of this CAR is depicted in FIG. 3, with the various domains indicated.

To assess impact on tumor growth, NSG male mice bearing PC-3 (FIG. 2B) and DU145 (FIG. 2C) tumors over-expressing human PSCA were treated with a single intravenous dose of 1, 2.5 or 5×10⁶ PSCA(ΔCH2)BBζ CAR or 5×10⁶ Mock (untransduced) T cells, and tumor volume was monitored by caliper measurement. To asses CD3, PD-1 and PD-L1 expression, PC-3-PSCA (FIG. 2D) and DU145-PSCA (FIG. 2E) tumors were stained for CD3 (human T cells), PD-1, and PD-L1 expression by immunohistochemistry, and representative images (20× magnification) are shown from Mock (untransduced) and PSCA (ΔCH2)BBζ CAR T cell-treated mice.

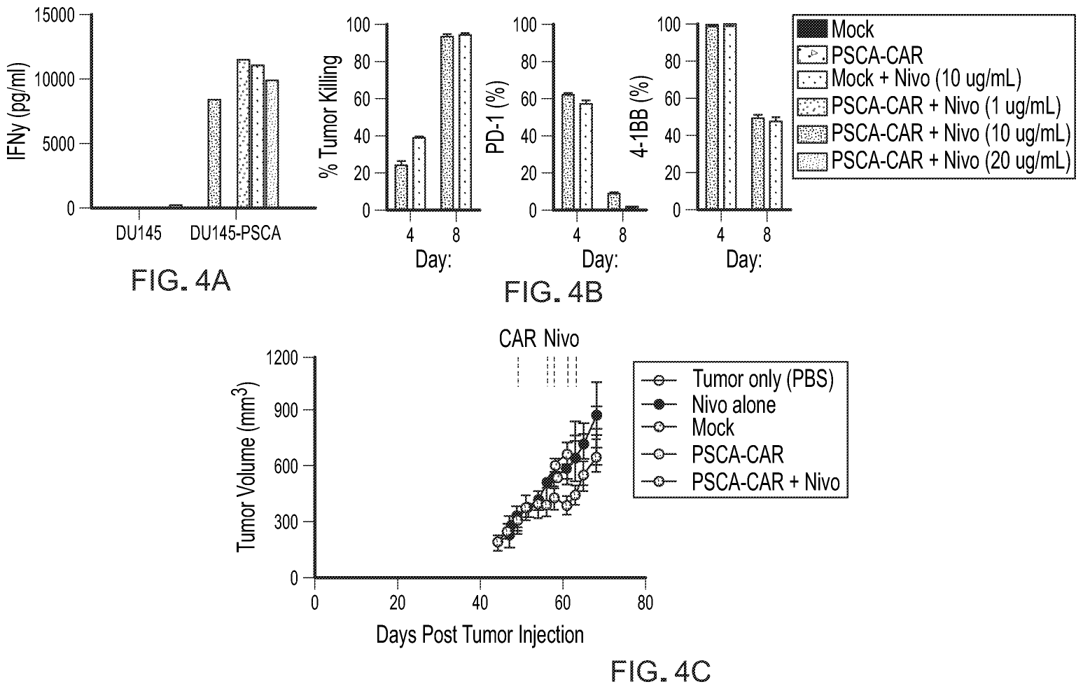

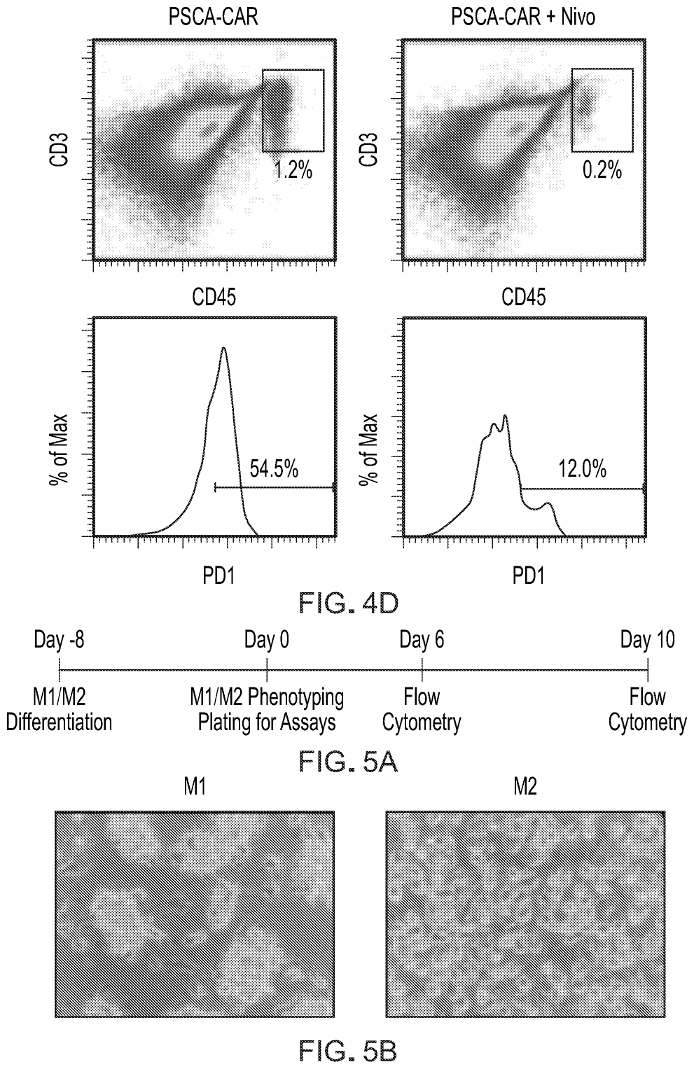

The impact of the anti-human PD-1 antibody Nivolumab and PSCA (ΔCH2)BBζ CAR T was examined by measuring IFNγ production in Mock (untransduced) or PSCA-CAR T cells cultured overnight with DU145 or DU145-PSCA tumor cells in the presence or absence of indicated concentrations of Nivolumab. As can be seen in FIG. 4A, IFNγ production was increased at 1 μg/ml Nivolumab, but decreased at higher concentrations. DU145-PSCA tumor killing, PD-1 expression and 4-1BB expression in PSCA-CAR T cells following a 4 and 8 day co-culture in the presence or absence of Nivolumab was also measured. The results of this analysis are shown in FIG. 4B. NSG male mice bearing DU145-PSCA tumors were treated with a single intravenous dose of 1×10⁶ PSCA(ΔCH2)BBζ CAR or Mock (untransduced) T cells, and treated with 4 doses with 100 ug Nivolumab, and tumor volume was monitored by caliper measurement. The results of this study are shown in FIG. 2C. In addition, both the percentage of human CD3+ cells and PD-1 expression in tumor-infiltrating T cells from mice treated with PSCA-CAR T cells alone or with Nivolumab was assessed (FIG. 2D).

The impact of M1 and M2 macrophages on PD-1 expression and PSCA-CAR T cell mediated anti-tumor responses in vitro was examined. Exposure of human monocytes to Th1 response-promoting IFN-γ or tumor necrosis factor alpha as well as the endotoxin lipopolysaccharide (sometimes referred to as “classical activation”) leads to M1 macrophages that function to produce pro-inflammatory mediators that provide host protection against bacteria and viruses. Human monocytes can also be differentiated to M2 macrophages by encountering Th2 response-promoting cytokines such as interleukin-10 and transforming growth factor-β (sometimes referred to as “alternative activation”). M2 macrophages express high levels of CD206 (mannose receptor) and CD163, produce low levels of pro-inflammatory cytokines, and promote wound healing and matrix remodeling.

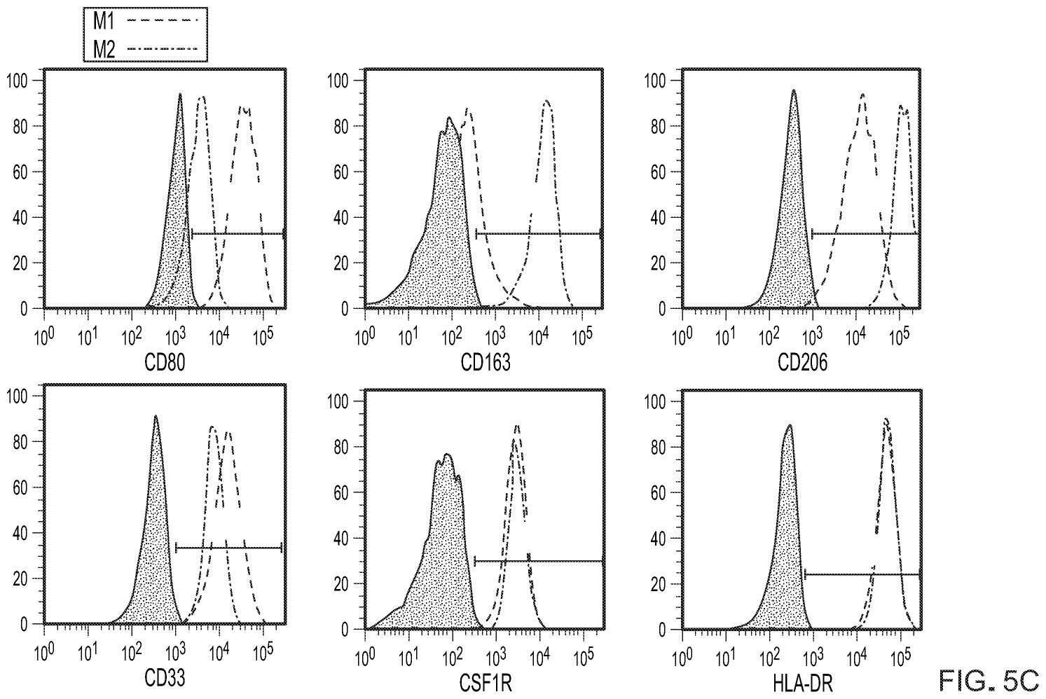

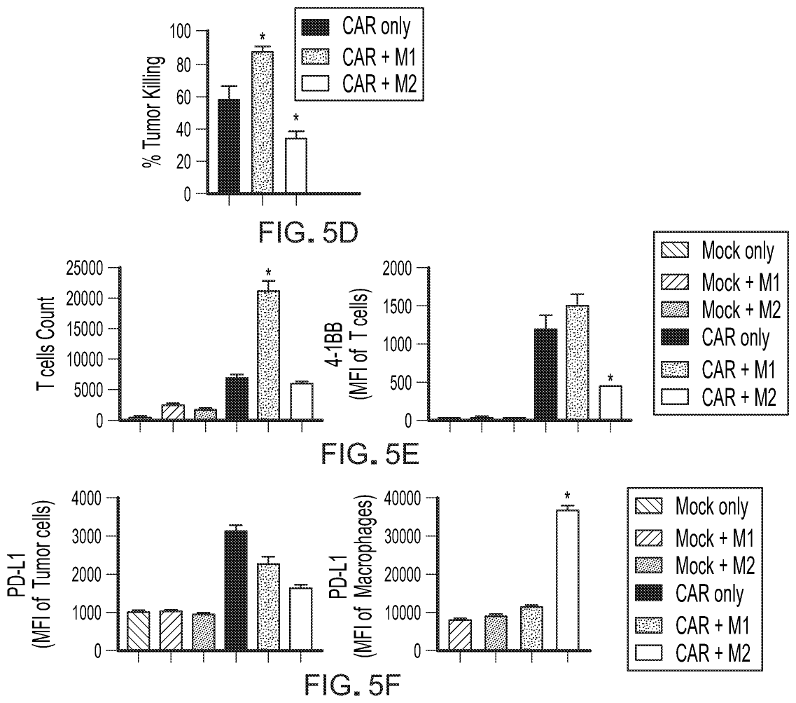

FIG. 5A is a schematic depiction of the assay used. The M1 and M2 macrophages were differentiated as described in Zarif et al. (Biotechniques 61:33, 2016). Macrophages, CAR T cells, and tumors were co-cultured for 6 or 10 days, and evaluated for functional assays using flow cytometry. Representative bright field images of M1 and M2 macrophages are shown in FIG. 5B and the phenotypes of M1 and M2 macrophages as assessed by certain cell surface markers are shown in FIG. 5C. PSCA(ΔCH2)BBζ CAR T cell killing of DU145-PSCA tumor cells in the presence or absence of M1 or M2 macrophages was assessed and the results are shown in FIG. 5D. PSCA-CAR T cell proliferation and activation (indicated by expression of 4-1BB) in the presence or absence of M1 and M2 macrophages is shown in FIG. 5E. Finally, PD-L1 expression in DU145-PSCA tumor cells and M1 or M2 macrophages following co-culture with Mock or PSCA-CAR T cells was assessed and the results of the study are shown in FIG. 5F. These studies show that M2 macrophages inhibit PSCA(ΔCH2)BBζ CAR T cell killing of DU145-PSCA and induce PDL-1 expression.

### Example 3: Vector for Knock-Down of PD-1, TIM3 and LAG3 Expression

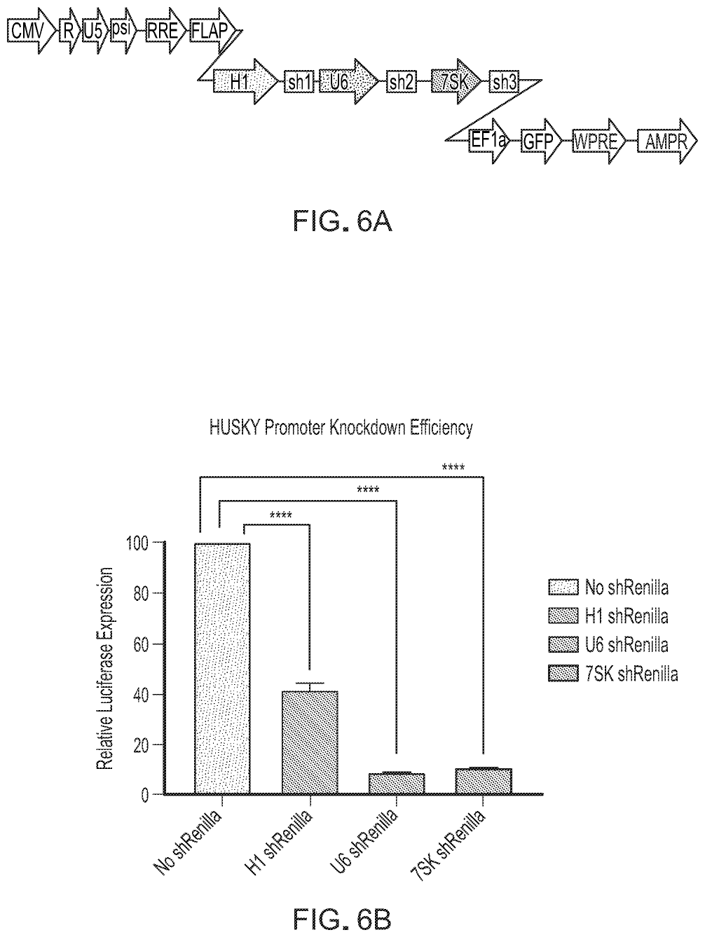

To create a vector useful for reducing expression of certain checkpoint inhibitors in cells expressing a CAR. shRNA sequences targeting PD-1, TIM-3, and LAG-3 were designed and used to create a cassette that can be installed in a lentiviral vector. Several potential shRNAs for each of PD-1, TIM-3, and LAG-3 were created and assessed. The shRNA are expressed under the control of three independent DNA Polymerase III promoters: the H1 promoter, the U6 promoter, and the 7SK promoter. The lentiviral vector has a pHIV7-backbone, a GFP reporter, and the H1, U6, and 7SK promoter cassette (referred to as “HUSKY”) with digestion sites for shRNAs following each promoter. The vector is schematically depicted in FIG. 6A and the sequence of the expression cassette without shRNA inserts in FIG. 9.

In order to assess each promoter in the cassette, HEK293T cells were co-transfected with a dual luciferase plasmid (Firefly and Renilla) and one of three HUSKY cassette vectors, each containing shRenilla driven by the H1 promoter, the U6 promoter, or the 7SK promoter. Empty HUSKY was used as a negative control. The results of this study are shown in FIG. 6B.

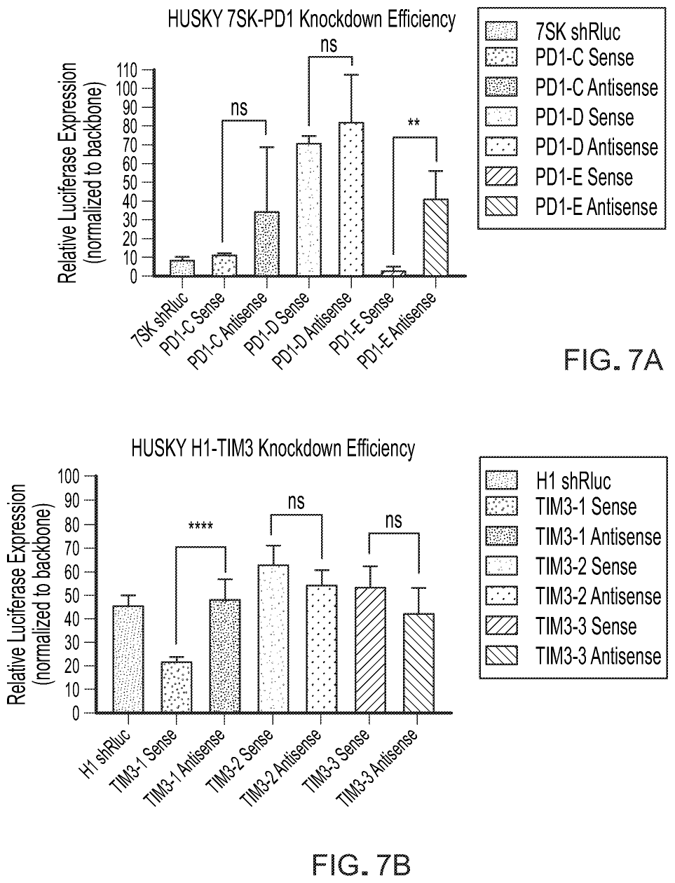

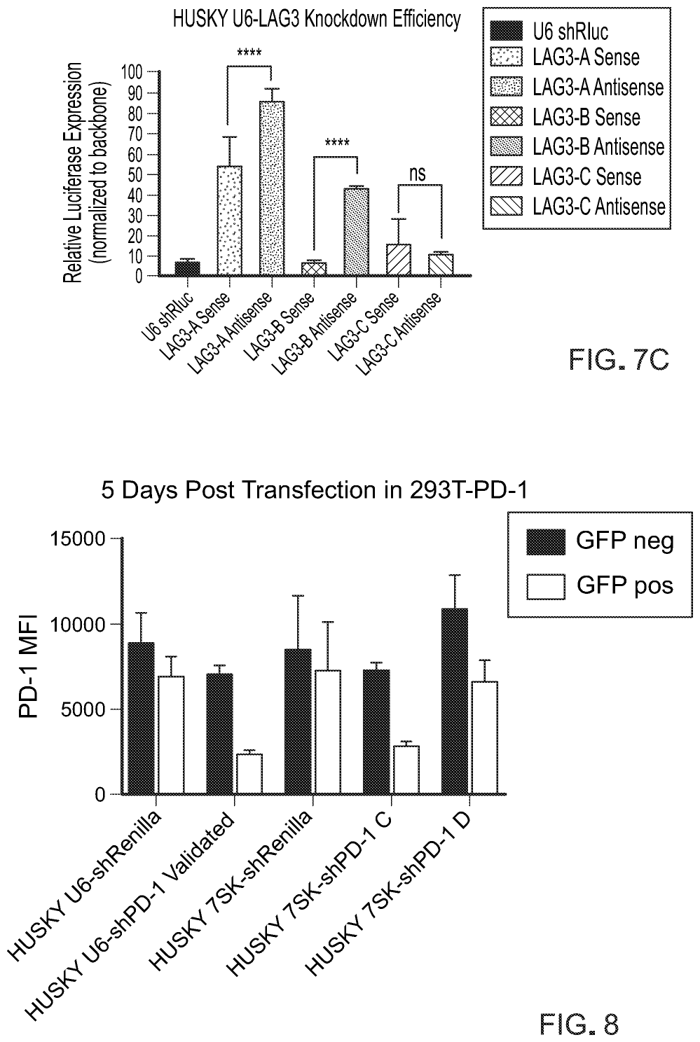

For each of the three targets, three different shRNA sequences were designed: PD1-C, -D and -E; TIM3-1, -2 and -3; and LAG3-A, -B and -C. HEK293T cells were co-transfected with a dual luciferase plasmid containing either the 100 bp sense or antisense targets for each shRNA in the Renilla 3′UTR and a HUSKY vector containing one of three candidate 7SK-driven shPD1, H1-driven shTIM3, or U6-driven shLAG3. HUSKY-shRenilla vectors for each promoter were used as positive controls and empty HUSKY as a negative control. Sense and antisense target strand knockdown was assessed. The results are presented in FIG. 7A-C. The shRNA sequences are in Table 4.

TABLE 4
snRNA Sequences

| Target | Name | Sequence |
|---|---|---|
| PD-1 | PD1-C | CCAACACATCGGAGAGCTTCGTTGTGTACGAAG |
| | | CTCTCCGATGTGTTGG (SEQ ID NO: 12) |
| | PD1-D | GAGTATGCCACCATTGTCTTTTTGTGTAAAAGA |
| | | CAATGGTGGCATACTC (SEQ ID NO: 13) |
| | PDI-E | CGTCCAGCTCCCTGAATCTCTTTGTGTAAGAGA |
| | | TTCAGGGAGCTGGACG (SEQ ID NO: 14) |
| TIM3 | TIM3-1 | CGTGGACCAAACTGAAGCTATTTGTGTAATAGC |
| | | TTCAGTTTGGTCCACG (SEQ ID NO: 15) |
| | TIM3-2 | GCACTGAACTTAAACAGGCATTTGTGTAATGCC |
| | | TGTTTAAGTTCAGTGC (SEQ ID NO: 16) |
| | TIM3-3 | CAAATGCAGTAGCAGAGGGAATTGTGTATTCCC |
| | | TCTGCTACTGCATTTG (SEQ ID NO: 17) |
| LAG3 | LAG3-A | GTGACTGGAGCCTTTGGCTTTTTGTGTAAAAGC |
| | | CAAAGGCTCCAGTCAC (SEQ ID NO: 18) |
| | LAG3-B | GGATCTCAGCCTTCTGCGAAGTTGTGTACTTCG |
| | | CAGAAGGCTGAGATCC (SEQ ID NO: 19) |
| | LAG3-C | GCAAGATAGAGGAGCTGGAGCTTGTGTAGCTCC |
| | | AGCTCCTCTATCTTGC (SEQ ID NO: 20) |

To assess knock-down of PD-1 by HUSKY shRNA constructs, PD-1 and GFP were overexpressed in 293T cells by lentivirus transduction, and double positive cells were obtained by FACS. HEK 293T-PD-1 cells were used to screen shRNA candidates against PD-1 by Lipofectamine transfection of plasmids with shRNA candidates on day 0. On day 5, PD-1 expression was evaluated by flow cytometry. The results of this assessment are presented in FIG. 8.

Human PD-1 (Genbank NM_005018) mRNA (SEQ ID NO: 25):

| | | | | | | |
|---|---|---|---|---|---|---|
| 1 | gctcacctcc | gcctgagcag | tggagaaggc | ggcactctgg | tggggctgct | ccaggcatgc |
| 61 | agatcccaca | ggcgccctgg | ccagtcgtct | gggcggtgct | acaactgggc | tggcggccag |
| 121 | gatggttctt | agactcccca | gacaggccct | ggaacccccc | caccttctcc | ccagccctgc |
| 181 | tcgtggtgac | cgaaggggac | aacgccacct | tcacctgcag | cttctccaac | acatcggaga |
| 241 | gcttcgtgct | aaactggtac | cgcatgagcc | ccagcaacca | gacggacaag | ctggccgcct |
| 301 | tccccgagga | ccgcagccag | cccggccagg | actgccgctt | ccgtgtcaca | caactgccca |
| 361 | acgggcgtga | cttccacatg | agcgtggtca | gggcccggcg | caatgacagc | ggcacctacc |
| 421 | tctgtggggc | catctccctg | gcccccaagg | cgcagatcaa | agagagcctg | cgggcagagc |
| 481 | tcagggtgac | agagagaagg | gcagaagtgc | ccacagccca | ccccagcccc | tcacccaggc |
| 541 | cagccggcca | gttccaaacc | ctggtggttg | gtgtcgtggg | cggcctgctg | ggcagcctgg |
| 601 | tgctgctagt | ctgggtcctg | gccgtcatct | gctcccgggc | cgcacgaggg | acaataggag |
| 661 | ccaggcgcac | cggccagccc | ctgaaggagg | acccctcagc | cgtgcctgtg | ttctctgtgg |
| 721 | actatgggga | gctggatttc | cagtggcgag | agaagacccc | ggagcccccc | gtgccctgtg |
| 781 | tccctgagca | gacggagtat | gccaccattg | tctttcctag | cggaatgggc | acctcatccc |
| 841 | ccgcccgcag | gggctcagct | gacggccctc | ggagtgccca | gccactgagg | cctgaggatg |
| 901 | gacactgctc | ttggcccctc | tgaccggctt | ccttggccac | cagtgttctg | cagaccctcc |
| 961 | accatgagcc | cgggtcagcg | catttcctca | ggagaagcag | gcagggtgca | ggccattgca |
| 1021 | ggccgtccag | gggctgagct | gcctgggggc | gaccggggct | ccagcctgca | cctgcaccag |
| 1081 | gcacagcccc | accacaggac | tcatgtctca | atgcccacag | tgagcccagg | cagcaggtgt |
| 1141 | caccgtcccc | tacagggagg | gccagatgca | gtcactgctt | caggtcctgc | cagcacagag |
| 1201 | ctgcctgcgt | ccagctccct | gaatctctgc | tgctgctgct | gctgctgctg | ctgctgcctg |
| 1261 | cggcccgggg | ctgaaggcgc | cgtggccctg | cctgacgccc | cggagcctcc | tgcctgaact |
| 1321 | tgggggctgg | ttggagatgg | ccttggagca | gccaaggtgc | ccctggcagt | ggcatcccga |
| 1381 | aacgccctgg | acgcagggcc | caagactggg | cacaggagtg | ggaggtacat | ggggctgggg |
| 1441 | actccccagg | agttatctgc | tccctgcagg | cctagagaag | tttcagggaa | ggtcagaaga |
| 1501 | gctcctggct | gtggtgggca | gggcaggaaa | cccctccacc | tttacacatg | cccaggcagc |
| 1561 | acctcaggcc | ctttgtgggg | cagggaagct | gaggcagtaa | gcgggcaggc | agagctggag |
| 1621 | gcctttcagg | cccagccagc | actctggcct | cctgccgccg | cattccaccc | cagcccctca |
| 1681 | caccactcgg | gagagggaca | tcctacggtc | ccaaggtcag | gagggcaggg | ctggggttga |
| 1741 | ctcaggcccc | tcccagctgt | ggccacctgg | gtgttgggag | ggcagaagtg | caggcaccta |
| 1801 | gggcccccca | tgtgcccacc | ctgggagctc | tccttggaac | ccattcctga | aattatttaa |
| 1861 | aggggttggc | cgggctccca | ccagggcctg | ggtgggaagg | tacaggcgtt | cccccggggc |
| 1921 | ctagtacccc | cgccgtggcc | tatccactcc | tcacatccac | acactgcacc | cccactcctg |
| 1981 | gggcagggcc | accagcatcc | aggcggccag | caggcacctg | agtggctggg | acaagggatc |
| 2041 | ccccttccct | gtggttctat | tatattataa | ttataattaa | atatgagagc | atgctaa |

Human TIM3 (Genbank NM_032782) mRNA (SEQ ID NO: 26)

| | | | | | | |
|---|---|---|---|---|---|---|
| 1 | atttggagag | ttaaaactgt | gcctaacaga | ggtgtcctct | gacttttctt | ctgcaagctc |
| 61 | catgttttca | catcttccct | ttgactgtgt | cctgctgctg | ctgctgctac | tacttacaag |
| 121 | gtcctcagaa | gtggaataca | gagcggaggt | cggtcagaat | gcctatctgc | cctgcttcta |
| 181 | caccccagcc | gccccaggga | acctcgtgcc | cgtctgctgg | ggcaaaggag | cctgtcctgt |
| 241 | gtttgaatgt | ggcaacgtgg | tgctcaggac | tgatgaaagg | gatgtgaatt | attggacatc |
| 301 | cagatactgg | ctaaatgggg | atttccgcaa | aggagatgtg | tccctgacca | tagagaatgt |
| 361 | gactctagca | gacagtggga | tctactgctg | ccggatccaa | atcccaggca | taatgaatga |
| 421 | tgaaaaattt | aacctgaagt | tggtcatcaa | accagccaag | gtcacccctg | caccgactcg |
| 481 | gcagagagac | ttcactgcag | cctttccaag | gatgcttacc | accaggggac | atggcccagc |
| 541 | agagacacag | acactgggga | gcctccctga | tataaatcta | acacaaatat | ccacattggc |
| 601 | caatgagtta | cgggactcta | gattggccaa | tgacttacgg | gactctggag | caaccatcag |
| 661 | aataggcatc | tacatcggag | cagggatctg | tgctgggctg | gctctggctc | ttatcttcgg |
| 721 | cgctttaatt | ttcaaatggt | attctcatag | caaagagaag | atacagaatt | taagcctcat |
| 781 | ctctttggcc | aacctccctc | cctcaggatt | ggcaaatgca | gtagcagagg | gaattcgctc |
| 841 | agaagaaaac | atctatacca | ttgaagagaa | cgtatatgaa | gtggaggagc | ccaatgagta |
| 901 | ttattgctat | gtcagcagca | ggcagcaacc | ctcacaacct | ttgggttgtc | gctttgcaat |
| 961 | gccatagatc | caaccacctt | atttttgagc | ttggtgtttt | gtctttttca | gaaactatga |
| 1021 | gctgtgtcac | ctgactggtt | ttggaggttc | tgtccactgc | tatggagcag | agttttccca |
| 1081 | ttttcagaag | ataatgactc | acatgggaat | tgaactggga | cctgcactga | acttaaacag |
| 1141 | gcatgtcatt | gcctctgtat | ttaagccaac | agagttaccc | aacccagaga | ctgttaatca |
| 1201 | tggatgttag | agctcaaacg | ggcttttata | tacactagga | attcttgacg | tggggtctct |
| 1261 | ggagctccag | gaaattcggg | cacatcatat | gtccatgaaa | cttcagataa | actagggaaa |
| 1321 | actgggtgct | gaggtgaaag | cataactttt | ttggcacaga | aagtctaaag | gggccactga |
| 1381 | ttttcaaaga | gatctgtgat | ccctttttgt | tttttgtttt | tgagatggag | tcttgctctg |
| 1441 | ttgcccaggc | tggagtgcaa | tggcacaatc | tcggctcact | gcaagctccg | cctcctgggt |
| 1501 | tcaagcgatt | ctcctgcctc | agcctcctga | gtggctggga | ttacaggcat | gcaccaccat |
| 1561 | gcccagctaa | tttgttgtat | ttttagtaga | gacagggttt | caccatgttg | gccagtgtgg |
| 1621 | tctcaaactc | ctgacctcat | gatttgcctg | cctcggcctc | ccaaagcact | gggattacag |
| 1681 | gcgtgagcca | ccacatccag | ccagtgatcc | ttaaaagatt | aagagatgac | tggaccaggt |
| 1741 | ctaccttgat | cttgaagatt | cccttggaat | gttgagattt | aggcttattt | gagcactgcc |
| 1801 | tgcccaactg | tcagtgccag | tgcatagccc | ttcttttgtc | tcccttatga | agactgccct |
| 1861 | gcagggctga | gatgtggcag | gagctcccag | ggaaaaacga | agtgcatttg | attggtgtgt |
| 1921 | attggccaag | ttttgcttgt | tgtgtgcttg | aaagaaaata | tctctgacca | acttctgtat |
| 1981 | tcgtggacca | aactgaagct | atatttttca | cagaagaaga | agcagtgacg | gggacacaaa |
| 2041 | ttctgttgcc | tggtggaaag | aaggcaaagg | ccttcagcaa | tctatattac | cagcgctgga |
| 2101 | tcctttgaca | gagagtggtc | cctaaactta | aatttcaaga | cggtataggc | ttgatctgtc |
| 2161 | ttgcttattg | ttgccccctg | cgcctagcac | aattctgaca | cacaattgga | acttactaaa |
| 2221 | aatttttttt | tactgtt | | | | |

Human LAG3 (Genbank NM_002286) mRNA (SEQ ID NO: 27)

| | | | | | | |
|---|---|---|---|---|---|---|
| 1 | agagaccagc | agaacggcat | cccagccacg | acggccactt | tgctctgtct | gctctccgcc |
| 61 | acggccctgc | tctgttccct | gggacacccc | cgcccccacc | tcctcaggct | gcctgatctg |
| 121 | cccagctttc | cagctttcct | ctggattccg | gcctctggtc | atccctcccc | accctctctc |
| 181 | caaggccctc | tcctggtctc | ccttcttcta | gaaccccttc | ctccacctcc | ctctctgcag |
| 241 | aacttctcct | ttacccccca | ccccccacca | ctgccccctt | tccttttctg | acctcctttt |
| 301 | ggagggctca | gcgctgccca | gaccatagga | gagatgtggg | aggctcagtt | cctgggcttg |
| 361 | ctgtttctgc | agccgctttg | ggtggctcca | gtgaagcctc | tccagccagg | ggctgaggtc |
| 421 | ccggtggtgt | gggcccagga | gggggctcct | gcccagctcc | cctgcagccc | cacaatcccc |
| 481 | ctccaggatc | tcagccttct | gcgaagagca | ggggtcactt | ggcagcatca | gccagacagt |
| 541 | ggcccgcccg | ctgccgcccc | cggccatccc | ctggcccccg | gccctcaccc | ggcggcgccc |
| 601 | tcctcctggg | ggcccaggcc | ccgccgctac | acggtgctga | gcgtgggtcc | cggaggcctg |
| 661 | cgcagcggga | ggctgcccct | gcagccccgc | gtccagctgg | atgagcgcgg | ccggcagcgc |
| 721 | ggggacttct | cgctatggct | gcgcccagcc | cggcgcgcgg | acgccggcga | gtaccgcgcc |
| 781 | gcggtgcacc | tcagggaccg | cgccctctcc | tgccgcctcc | gtctgcgcct | gggccaggcc |
| 841 | tcgatgactg | ccagcccccc | aggatctctc | agagcctccg | actgggtcat | tttgaactgc |
| 901 | tccttcagcc | gccctgaccg | cccagcctct | gtgcattggt | tccggaaccg | gggccagggc |
| 961 | cgagtccctg | tccgggagtc | cccccatcac | cacttagcgg | aaagcttcct | cttcctgccc |
| 1021 | caagtcagcc | ccatggactc | tgggccctgg | ggctgcatcc | tcacctacag | agatggcttc |
| 1081 | aacgtctcca | tcatgtataa | cctcactgtt | ctgggtctgg | agcccccaac | tcccttgaca |
| 1141 | gtgtacgctg | gagcaggttc | cagggtgggg | ctgccctgcc | gcctgcctgc | tggtgtgggg |
| 1201 | acccggtctt | tcctcactgc | caagtggact | cctcctgggg | gaggccctga | cctcctggtg |
| 1261 | actggagaca | atggcgactt | tacccttcga | ctagaggatg | tgagccaggc | ccaggctggg |
| 1321 | acctacacct | gccatatcca | tctgcaggaa | cagcagctca | atgccactgt | cacattggca |
| 1381 | atcatcacag | tgactcccaa | atcctttggg | tcacctggat | ccctggggaa | gctgctttgt |
| 1441 | gaggtgactc | cagtatctgg | acaagaacgc | tttgtgtgga | gctctctgga | caccccatcc |
| 1501 | cagaggagtt | tctcaggacc | ttggctggag | gcacaggagg | cccagctcct | ttcccagcct |
| 1561 | tggcaatgcc | agctgtacca | gggggagagg | cttcttggag | cagcagtgta | cttcacagag |
| 1621 | ctgtctagcc | caggtgccca | acgctctggg | agagccccag | gtgccctccc | agcaggccac |
| 1681 | ctcctgctgt | ttctcatcct | tggtgtcctt | tctctgctcc | ttttggtgac | tggagccttt |
| 1741 | ggctttcacc | tttggagaag | acagtggcga | ccaagacgat | tttctgcctt | agagcaaggg |
| 1801 | attcaccctc | cgcaggctca | gagcaagata | gaggagctgg | agcaagaacc | ggagccggag |
| 1861 | ccggagccgg | aaccggagcc | cgagcccgag | cccgagccgg | agcagctctg | acctggagct |
| 1921 | gaggcagcca | gcagatctca | gcagcccagt | ccaaataaac | tccctgtcag | cagcaa |

1. A nucleic acid vector comprising an shRNA sequence targeted to an mRNA encoding a checkpoint inhibitor selected from the group consisting of PD-1, TIM3 and LAG3 and a nucleic acid sequence encoding a chimeric antigen receptor (CAR).

2. The nucleic acid vector of claim 1, wherein the CAR targets an antigen selected from the group consisting of IL-13Ra, HER2, CD19 and PSCA.

3. The nucleic acid vector of claim 1, wherein each shRNA sequence is operably linked to a promoter.

4. The nucleic acid vector of claim 3, wherein each shRNA sequence is operably linked to the same promoter.

5. The nucleic acid vector of claim 1, wherein the vector comprises two or more different shRNA nucleic acid sequences targeted to the same mRNA encoding a checkpoint inhibitor.

6. The nucleic acid vector of claim 1, wherein each shRNA sequence is targeted to a different mRNA encoding a checkpoint inhibitor.

7. The nucleic acid vector of claim 1, wherein the vector comprises an shRNA sequence targeted to an mRNA encoding PD-1 and an shRNA sequence targeted to an mRNA encoding TIM3.

8. The nucleic acid vector of claim 1, wherein the vector comprises an shRNA sequence targeted to an mRNA encoding PD-1 and an shRNA sequence targeted to an mRNA encoding LAG3.

9. The nucleic acid vector of claim 1, wherein the vector comprises an shRNA sequence targeted to an mRNA encoding TIM3 and an shRNA sequence targeted to an mRNA encoding LAG3.

10. The nucleic acid vector of claim 3, wherein each shRNA sequence is operably linked to a different promoter.

11. The nucleic acid vector of claim 3, comprising two or more different shRNA sequences that are operably linked to the same promoter.

12. The nucleic acid vector of claim 3, wherein the promoter is selected from the group consisting of an H1 DNA POL III promoter, an U6 DNA POL III promoter, and a 75K DNA POL III.

13. The nucleic acid vector of claim 1, wherein the shRNA targeted to an mRNA encoding PD-1 is targeted to SEQ ID NO:25.

14. The nucleic acid vector of claim 1, wherein the shRNA targeted to an mRNA encoding TIM3 is targeted to SEQ ID NO:26.

15. The nucleic acid vector of claim 1, wherein the shRNA targeted to an mRNA encoding LAG3 is targeted to SEQ ID NO:27.

16. The nucleic acid vector of claim 1, wherein the shRNA targeted to PD-1 comprises a nucleic acid sequence selected from the group consisting of SEQ ID Nos 12-14.

17. The nucleic acid vector of claim 1, wherein the shRNA targeted to TIM3 comprises a nucleic acid sequence selected from the group consisting of SEQ ID Nos: 15-17.

18. The nucleic acid vector of claim 1, wherein the shRNA targeted to LAG3 comprises a nucleic acid sequence selected from the group consisting of SEQ ID Nos: 18-20.

19. The nucleic acid vector of claim 1, wherein each shRNA sequence comprises:
sense sequence and an antisense sequence, wherein the sense and the antisense sequence, wherein the sense sequence comprises a nucleotide sequence identical to a target sequence in an mRNA encoding a checkpoint inhibitor.

20. The nucleic acid vector of claim 1, wherein the vector is a lentiviral vector.

21. A nucleic acid vector comprising an H1 DNA POL III promoter, an U6 DNA POL III promoter, and a 75K DNA POL III promoter, where each promoter is operably linked to an shRNA nucleic acid sequence targeted to an mRNA encoding a checkpoint inhibitor.

22. The nucleic acid vector of claim 21, wherein each shRNA nucleic acid sequence is targeted to the same mRNA encoding a checkpoint inhibitor.

23. The nucleic acid vector of claim 21, wherein each shRNA is targeted to a different mRNA encoding a checkpoint inhibitor.

24. The nucleic acid vector of claim 21, wherein the checkpoint inhibitor is selected from the group consisting of PD-1, TIM3 and LAG3.

25. The nucleic acid vector of claim 21, wherein the shRNA targeted to an mRNA encoding PD-1 is targeted to SEQ ID NO:25.

26. The nucleic acid vector of claim 21, wherein the shRNA targeted to an mRNA encoding TIM3 is targeted to SEQ ID NO:26.

27. The nucleic acid vector of claim 21, wherein the shRNA targeted to an mRNA encoding LAG3 is targeted to SEQ ID NO:27.

28. The nucleic acid vector of claim 25, wherein the shRNA targeted to PD-1 comprises a nucleic acid sequence selected from the group consisting of SEQ ID Nos 12-14.

29. The nucleic acid vector of claim 26, wherein the shRNA targeted to TIM3 comprises a nucleic acid sequence selected from the group consisting of SEQ ID Nos: 15-17.

30. The nucleic acid vector of claim 27, wherein the shRNA targeted to LAG3 comprises a nucleic acid sequence selected from the group consisting of SEQ ID Nos: 18-20.

31. A nucleic acid vector of claim 1, wherein the vector comprises the nucleotide sequence of SEQ ID NO:24.

32. The nucleic acid vector of claim 1, wherein each shRNA sequence comprises:
sense sequence and an antisense sequence, wherein the sense and the antisense sequence, wherein the sense sequence comprises a nucleotide sequence identical to a target sequence in an mRNA encoding a checkpoint inhibitor.

33. The nucleic acid vector of claim 1, wherein the vector is a lentiviral vector.

34. The nucleic acid vector of claim 21, wherein the vector further comprises a nucleotide sequence encoding a chimeric antigen receptor.

35. The nucleic acid vector of claim 34, wherein the CAR targets an antigen selected from the group consisting of: PSCA, HER2 and CD19.

36. The nucleic acid vector of claim 44, wherein the CAR comprises, from amino to carboxy terminus: a targeting domain for targeting an antigen; a spacer domain; a transmembrane domain; a co-stimulatory domain; and a CD3ζ signaling domain.

37. A T cell harboring the nucleic acid vector of claim 1.

38. A method for reducing the expression of a checkpoint inhibitor in T cell, the method comprising introducing the nucleic acid vector of claim 1 into the T cell.


SEQUENCE LISTING (59 sequences)

SEQ ID NO: 1-11 - skipped (11 entries)

SEQ ID NO: 12 - DNA, 49 bp, Artificial Sequence
  PD1-C
ccaacacatcggagagcttcgttgtgtacgaagctctccgatgtgttgg

SEQ ID NO: 13 - DNA, 49 bp, Artificial Sequence
  PD1-D
gagtatgccaccattgtctttttgtgtaaaagacaatggtggcatactc

SEQ ID NO: 14 - DNA, 49 bp, Artificial Sequence
  PD1-E
cgtccagctccctgaatctctttgtgtaagagattcagggagctggacg

SEQ ID NO: 15 - DNA, 49 bp, Artificial Sequence
  TIM3-1
cgtggaccaaactgaagctatttgtgtaatagcttcagtttggtccacg

SEQ ID NO: 16 - DNA, 49 bp, Artificial Sequence
  TIM3-2
gcactgaacttaaacaggcatttgtgtaatgcctgtttaagttcagtgc

SEQ ID NO: 17 - DNA, 49 bp, Artificial Sequence
  TIM3-3
caaatgcagtagcagagggaattgtgtattccctctgctactgcatttg

SEQ ID NO: 18 - DNA, 49 bp, Artificial Sequence
  LAG3-A
gtgactggagcctttggctttttgtgtaaaagccaaaggctccagtcac

SEQ ID NO: 19 - DNA, 49 bp, Artificial Sequence
  LAG3-B
ggatctcagccttctgcgaagttgtgtacttcgcagaaggctgagatcc

SEQ ID NO: 20 - DNA, 49 bp, Artificial Sequence
  LAG3-C
gcaagatagaggagctggagcttgtgtagctccagctcctctatcttgc

SEQ ID NO: 21 - DNA, 212 bp, Artificial Sequence
  H1
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagac

SEQ ID NO: 22 - DNA, 255 bp, Artificial Sequence
  U6
ggatccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtgg

SEQ ID NO: 23 - DNA, 240 bp, Artificial Sequence
  7SK
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtac

SEQ ID NO: 24 - DNA, 835 bp, Artificial Sequence
  HUSKY cassette without any inserts
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacgagatgcaggtagaattcaattgacctgcgtcgttttttggcgcgccggatccaaggtcgggcaggaaggatccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgagacggaattcgagatgcgtctcattttttaattaactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 25 - DNA, 2097 bp, Artificial Sequence
  Human PD-1 (Genbank NM_005018) mRNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 26 - DNA, 2237 bp, Artificial Sequence
  Human TIM3 (Genbank NM_032782) mRNA
atttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgtt

SEQ ID NO: 27 - DNA, 1976 bp, Artificial Sequence
  Human LAG3 (Genbank NM_002286) mRNA
agagaccagcagaacggcatcccagccacgacggccactttgctctgtctgctctccgccacggccctgctctgttccctgggacacccccgcccccacctcctcaggctgcctgatctgcccagctttccagctttcctctggattccggcctctggtcatccctccccaccctctctccaaggccctctcctggtctcccttcttctagaaccccttcctccacctccctctctgcagaacttctcctttaccccccaccccccaccactgccccctttccttttctgacctccttttggagggctcagcgctgcccagaccataggagagatgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctgacctggagctgaggcagccagcagatctcagcagcccagtccaaataaactccctgtcagcagcaa

SEQ ID NO: 28 - protein, 236 aa, Artificial Sequence
  PSCA targeting scFv
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 29 - protein, 247 aa, Artificial Sequence
  FMC63 scFv
IPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 31 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 32 - protein, 10 aa, Artificial Sequence
  Linker
GGGSSGGGSG

SEQ ID NO: 33 - protein, 12 aa, Artificial Sequence
  IgG4 hinge (S->P) (S228P)
ESKYGPPCPPCP

SEQ ID NO: 34 - protein, 12 aa, Artificial Sequence
  IgG4 hinge
ESKYGPPCPSCP

SEQ ID NO: 35 - protein, 22 aa, Artificial Sequence
  IgG4 hinge (S228P)+ linker
ESKYGPPCPPCPGGGSSGGGSG

SEQ ID NO: 36 - protein, 39 aa, Artificial Sequence
  CD28 hinge
IEVMYPPPYLDNEKSNGTIIHVKGKHLCPSPLFPGPSKP

SEQ ID NO: 37 - protein, 48 aa, Artificial Sequence
  CD8 hinge-48aa
AKPTTTPAPRPPTPAPTIASQPLSLRPEACRPAAGGAVHTRGLDFACD

SEQ ID NO: 38 - protein, 45 aa, Artificial Sequence
  CD8 hinge-45aa
TTTPAPRPPTPAPTIASQPLSLRPEACRPAAGGAVHTRGLDFACD

SEQ ID NO: 39 - protein, 129 aa, Artificial Sequence
  IgG4(HL-CH3) (includes S228P in hinge)
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 229 aa, Artificial Sequence
  IgG4(L235E,N297Q)
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 229 aa, Artificial Sequence
  IgG4(S228P, L235E,N297Q)
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 107 aa, Artificial Sequence
  IgG4(CH3)
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 21 aa, Artificial Sequence
  CD3z
LCYLLDGILFIYGVILTALFL

SEQ ID NO: 44 - protein, 27 aa, Artificial Sequence
  CD28
FWVLVVVGGVLACYSLLVTVAFIIFWV

SEQ ID NO: 45 - protein, 28 aa, Artificial Sequence
  CD28(M)
MFWVLVVVGGVLACYSLLVTVAFIIFWV

SEQ ID NO: 46 - protein, 22 aa, Artificial Sequence
  CD4
MALIVLGGVAGLLLFIGLGIFF

SEQ ID NO: 47 - protein, 21 aa, Artificial Sequence
  CD8tm
IYIWAPLAGTCGVLLLSLVIT

SEQ ID NO: 48 - protein, 23 aa, Artificial Sequence
  CD8tm2
IYIWAPLAGTCGVLLLSLVITLY

SEQ ID NO: 49 - protein, 24 aa, Artificial Sequence
  CD8tm3
IYIWAPLAGTCGVLLLSLVITLYC

SEQ ID NO: 50 - protein, 27 aa, Artificial Sequence
  41BB
IISFFLALTSTALLFLLFFLTLRFSVV

SEQ ID NO: 51 - protein, 112 aa, Artificial Sequence
  CD3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 41 aa, Artificial Sequence
  CD28
RSKRSRLLHSDYMNMTPRRPGPTRKHYQPYAPPRDFAAYRS

SEQ ID NO: 53 - protein, 41 aa, Artificial Sequence
  CD28gg*
RSKRSRGGHSDYMNMTPRRPGPTRKHYQPYAPPRDFAAYRS

SEQ ID NO: 54 - protein, 42 aa, Artificial Sequence
  4-1BB
KRGRKKLLYIFKQPFMRPVQTTQEEDGCSCRFPEEEEGGCEL

SEQ ID NO: 55 - protein, 42 aa, Artificial Sequence
  OX40
ALYLLRRDQRLPPDAHKPPGGGSFRTPIQEEQADAHSTLAKI

SEQ ID NO: 56 - protein, 24 aa, Artificial Sequence
  ribosomal skip sequence
LEGGGEGRGSLLTCGDVEENPGPR

SEQ ID NO: 57 - protein, 354 aa, Artificial Sequence
  truncated EGFR
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 58 - protein, 129 aa, Artificial Sequence; identical to SEQ ID NO: 39
  linker

SEQ ID NO: 59 - protein, 526 aa, Artificial Sequence
  PSCAscFv-IgG4(HL-CH3)CD4tm-41BB-Zeta
MLLLVTSLLLCELPHPAFLLIPDIQLTQSPSTLSASVGDRVTITCSASSSVRFIHWYQQKPGKAPKRLIYDTSKLASGVPSRFSGSGSGTDFTLTISSLQPEDFATGMCSFRYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGGKPRRKNPQEGLYNELQKDKMAEAYSEIGMKGERRRGKGHDGLYQGLSTATKDTYDALHMQALPPR